(19) United States
(12) Patent Application Publication
(10) Pub. No.: US 2021/0405067 A1
(43) Pub. Date: Dec. 30, 2021

(54) COMPOSITION FOR DIAGNOSING MILD COGNITIVE IMPAIRMENT CONTAINING TonEBP ANTIBODY AS AN EFFECTIVE COMPONENT

(71) Applicants: INDUSTRY-ACADEMIC COOPERATION FOUNDATION GYEONGSANG NATIONAL UNIVERSITY, Gyeongsangnam-do (KR); INDUSTRY-ACADEMIC COOPERATION FOUNDATION, CHOSUN UNIVERSITY, Gwangju (KR)

(72) Inventors: Gu Seob ROH, Gyeongsangnam-do (KR); Jong Youl LEE, Gyeongsangnam-do (KR); Kyung Eun KIM, Gyeongsangnam-do (KR); Kun Ho LEE, Gwangju (KR); Eun-Ae JEONG, Gyeongsangbuk-do (KR)

(21) Appl. No.: 17/294,507

(22) PCT Filed: Sep. 11, 2019
(86) PCT No.: PCT/KR2019/011854

§ 371 Date: May 17, 2021

(30) Foreign Application Priority Data
Nov. 16, 2018 (KR) 10-2018-0141781

(51) Int. Cl. G01N 33/68

(52) U.S. Cl. CPC G01N 33/6896; G01N 2333/4703; G01N 2800/2814

(57) ABSTRACT

A method for diagnosing mild cognitive impairment according to an embodiment of the present disclosure includes obtaining a sample from a subject to be diagnosed, and measuring a level of tonicity-responsible enhancer binding protein (TonEBP) in the sample by using TonEBP antibody to determine if the measured level of the TonEBP is above a predetermined level. The measuring may further includes measuring a level of lipocalin-2 (LCN2) in the sample by using LCN2 antibody to determine if the measured level of the LCN2 is above a predetermined level.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2019/011854, filed Sep. 11, 2019, which claims priority to the benefit of Korean Patent Application No. 10-2018-0141781 filed in the Korean Intellectual Property Office on Nov. 16, 2018, the entire contents of which are incorporated herein by reference.

### BACKGROUND

### 1. Technical Field

The present invention relates to a composition for diagnosing mild cognitive impairment containing TonEBP antibody as an effective component.

### 2. Background Art

Cognitive brain impairment is clinically characterized by gradual decline in memory performance, cognitive function, reasoning function, executive function, planning function, judgment function, and emotional stability. The cognitive impairment gradually leads to severe intellectual disability and it may result from a broad range of disorders.

In particular, mild cognitive impairment (MCI) indicates pre-stage dementia in which people having MCI still maintain the ability of performing daily activities even though the cognitive function, particularly memory performance, is lower than people in the same age group. People showing the signs of MCI are recognized as a high-risk group with very high possibility of developing dementia.

MCI is a condition for which dementia can be detected at the earliest stage, and, from the recent finding that a new type of pharmaceutical for treating dementia works more efficiently at early stage than late stage, early diagnosis of MCI has very high clinical importance.

Risk of having MCI is affected by various factors. Obesity and type 2 diabetes rising in recent years are also one of the various risk factors and obesity is also regarded as a risk factor affecting brain structure. Many studies are made in recent years on the relationship between metabolic syndrome and dementia. As such, it is necessary to make early diagnosis of MCI for patients having obesity and diabetes, whose numbers are currently increasing at a steady pace.

Meanwhile, TonEBP (tonicity-responsive enhancer binding protein), which is also referred to as NFATS (nuclear factor of activated T cells 5), is a TonE-related dimer protein. NFATS/TonEBP stimulates the transcription of a synthase and a membrane transporter for organic osmolytes and it also stimulates the cellular accumulation of organic osmolytes. With the organic osmolytes, a change causing hypertonicity based on cellular volume and intracellular ionic strength is restored to normal level. In addition, expression of HSP70, which protects renal medullary cells against adverse effect exhibited high urea, is stimulated by NFATS/TonEBP. Thus, NFATS/TonEBP is an important regulator for many pathways in hyperosmotic kidney. It is also known that enhanced expression and higher activity of TonEBP are found in rheumatic arthritis, atherosclerosis, and diabetic nephropathy but the inflammatory disease development can be dramatically inhibited by reducing the activity of TonEBP just by 50%.

As a prior technique relating to diagnosis of MCI, a composition and a kit for diagnosing MCI including measuring the level of lipocalin 2 (LCN2) and a method of providing information for diagnosing MCI are described in Korean Patent Registration No. 1295019. However, so far there is no disclosure of a composition for diagnosing mild cognitive impairment containing TonEBP antibody as an effective component as it is described in the present invention.

### SUMMARY

The present invention is devised under the circumstances that are described above. Specifically, based on the finding that the expression of TonEBP is enhanced in the blood of a patient with diabetes and cognitive impairment and also in the blood of a diabetic mouse with cognitive impairment, a composition for diagnosing mild cognitive impairment containing TonEBP (tonicity-responsive enhancer binding protein) antibody specifically binding to TonEBP as an effective component is provided, a kit including the composition is prepared, a method for measuring the amount of TonEBP protein to provide information for diagnosing mild cognitive impairment including measuring the amount of TonEBP protein in a test sample isolated from an analyte by using the TonEBP antibody is provided, and the present invention is completed accordingly.

To achieve the purpose described above, the present invention provides a composition for diagnosing mild cognitive impairment containing TonEBP antibody specifically binding to TonEBP (tonicity-responsible enhancer binding protein) as an effective component.

The present invention further provides a kit for diagnosing mild cognitive impairment including the composition.

The present invention still further provides a method for measuring the amount of TonEBP protein to provide information for diagnosing mild cognitive impairment including measuring the amount of TonEBP in a test sample isolated from an analyte by using TonEBP antibody.

The present invention relates to a composition for diagnosing mild cognitive impairment containing TonEBP antibody as an effective component. Specifically, it is found that the expression of TonEBP protein and LCN2 protein is enhanced in the blood of a patient with diabetes and cognitive impairment who has been diagnosed with mild cognitive impairment. It is also found that a mouse induced to have obesity or obesity and diabetes is identified to have cognitive impairment and the expression of TonEBP protein is enhanced in the blood of the mouse. Since LCN2 protein is also enhanced in the blood in which TonEBP protein is enhanced, the present invention has an effect of allowing diagnosis and identification of a patient with mild cognitive impairment according to measurement of the level of protein concentration through detection of TonEBP protein or parallel detection of TonEBP and LCN2 proteins in the blood of a patient.

### BRIEF DESCRIPTION OF THE DRAWINGS

FIG. 1 shows samples of (A) K-MMSE test sheet and (B) SVLT-delay test sheet for testing cognitive function.

FIG. 2 shows the result of measuring the concentration of (A) LCN2 and (B) TonEBP proteins in blood serum of people selected via dementia selection and in-depth examination which have been carried out for the elderlies in dementia cohort as a subject. CTL represents a normal group, DM represents diabetic group, and DM-MCI represents diabetic group with mild cognitive impairment. * indicates that the expression of LCN2 or TonEBP protein in blood is enhanced in DM-MCI group compared to DM group, in which p<0.05.

FIG. 3 shows the result of measuring (A) the body weight of a mouse with induced obesity and diabetes and (B) the weight of each tissue of the mouse, and determining (C) fat level based on a photographic image. ND represents a normal diet group, HFD represents a group with obesity induced by high fat diet, and HFD+STZ represents a group with obesity and diabetes induced by high fat diet and a streptozotocin (STZ) treatment. * indicates that the weight has increased in significant sense in HFD group compared to ND group, in which p<0.05. † indicates that the weight has decreased in significant sense in HFD+STZ group compared to HFD group, in which p<0.05.

FIG. 4 shows the result of measuring the protein concentration of (A) TonEBP and (B) LCN2 in the blood of a mouse with induced obesity and diabetes, (C) correlation between LCN2 and TonEBP proteins in blood, and (D) correlation between the LCN2 concentration in blood and blood sugar. ND represents a normal diet group, HFD represents a group with obesity induced by high fat diet, and HFD+STZ represents a group with obesity and diabetes induced by high fat diet and a streptozotocin (STZ) treatment. * indicates that the protein concentration in blood has increased in significant sense in HFD group or HFD+STZ group compared to ND group, in which p<0.05. † indicates that the protein concentration in blood has increased in significant sense in HFD+STZ group compared to HFD group, in which p<0.05.

FIG. 5 shows the result of Morris water maze test of a mouse which has been induced to have obesity and diabetes, i.e., measurement of (A) escape latency, i.e., the time required for the animal to climb onto a round escape platform, (B) time in the target platform quadrant, i.e., after showing the location of a round escape platform to a mouse not able to climb onto the round escape platform even after 90 seconds, forcing the mouse, 5 days thereafter, to swim for 60 seconds by removing the round escape platform, the time for the animal to spend in the area of round escape platform, and (C) the number of the platform crossings, i.e., the number of the round escape platform crossings by the animal. In the figure, (D) shows the result of recording every behavior of a mouse by using a video tracking system. ND represents a normal diet group, HFD represents a group with obesity induced by high fat diet, and HFD+STZ represents a group with obesity and diabetes induced by high fat diet and a streptozotocin (STZ) treatment. * indicates that the time required for the animal to climb onto a round escape platform, the time for the animal to spend in the area of round escape platform, and the number of the round escape platform crossings by the animal have decreased in significant sense in HFD group or HFD+STZ group compared to ND group, in which p<0.05.

FIG. 6 shows the result of determining the expression of (A) TonEBP protein and (B) LCN2 protein in the hippocampal tissues of a mouse with impaired cognitive function, in which the animal has been induced to have obesity and diabetes. ND represents a normal diet group, HFD represents a group with obesity induced by high fat diet, and HFD+STZ represents a group with obesity and diabetes induced by high fat diet and a streptozotocin (STZ) treatment. Total represents the protein lysate of entire hippocampal tissues and Nu represents the lysate of nuclear proteins which have been isolated from the hippocampal tissues. * indicates that the protein concentration has increased in significant sense in HFD group or HFD+STZ group compared to ND group, in which p<0.05. † indicates that the protein concentration has increased in significant sense in HFD+STZ group compared to HFD group, in which p<0.05.

FIG. 7 shows the result of determining (A) blood glucose, (B) correlation between blood glucose and LCN2, (C) and concentration of LCN2 and TonEBP proteins in the hippocampal tissues of an ob/ob mouse, which is an animal model of obesity and Type 2 diabetes. WT represents a normal animal model having no mutation of leptin gene, and ob/ob mouse represents an animal model of obesity and Type 2 diabetes which has a mutation of leptin gene.

FIG. 8 shows the result of determining (A) the brain weight, (B) the ratio between brain weight and body weight of an ob/ob mouse, which is an animal model of obesity and Type 2 diabetes, (C) histological staining of a specimen of the brain tissues, and (D) quantification of the volume of the hippocampal tissues inside the dotted box. WT represents a normal animal model having no mutation of leptin gene, and ob/ob mouse represents an animal model of obesity and Type 2 diabetes, which has a mutation of leptin gene.

### DETAILED DESCRIPTION

The present invention relates to a composition for diagnosing mild cognitive impairment containing TonEBP antibody specifically binding to TonEBP (tonicity-responsible enhancer binding protein) as an effective component.

As described herein, the expression “mild cognitive impairment (MCI)” indicates a symptom characterized by a slight but measurable decline in cognitive abilities although it is not necessarily related with the onset of dementia. Although not necessarily, MCI may often develop into Alzheimer's disease.

The composition for diagnosing mild cognitive impairment may further contain, in addition to the aforementioned effective component, LCN2 antibody which specifically binds to LCN2 (lipocalin-2) protein, but it is not limited thereto.

The antibody of the present invention encompasses both the monoclonal antibody and polyclonal antibody.

The mild cognitive impairment may be a condition caused by metabolic disorder. Preferably, it may be a condition caused by obesity or diabetes, and more preferably a condition caused by diabetes resulting from obesity, but it is not limited thereto.

In order to improve the quickness and convenience of diagnosis, the composition for diagnosis of the present invention may be provided in immobilized form on a suitable carrier or support by using various methods that are well known. Examples of the suitable carrier or support include agarose, cellulose, nitrocellulose, dextran, sephadex, sepharose, liposome, carboxymethyl cellulose, polyacrylamide, polystyrene, gabbro, filter paper, ion exchange resin, plastic film, plastic tube, glass, polyamine-methyl vinyl-ether-maleic acid copolymer, amino acid copolymer, ethylene-maleic acid copolymer, nylon, cup, and flat pack. Examples of a solid substrate other than those include cell culture plate, ELISA plate, tube, and polymeric membrane. The support may have any possible shape such as globule (e.g., beads), barrel (e.g., inner wall of test tube or well), or planar shape (e.g., sheet or test strip).

The present invention further relates to a kit for diagnosing mild cognitive impairment including the aforementioned composition.

The kit for diagnosis may be provided in the form of a lateral flow assay kit that is based on immunochromatography to detect TonEBP protein in a test sample of blood serum, for example. The lateral flow assay kit may be provided by having a sample pad to which a test sample of blood serum is applied, a releasing pad coated with a probe antibody, a development membrane (e.g., nitrocellulose) or strip on which the test sample is separated after transfer and an antigen-antibody reaction occurs, and an absorption pad.

The present invention still further relates to a method for measuring an amount of TonEBP protein to provide information for diagnosing mild cognitive impairment including measuring the amount of TonEBP in a sample isolated from a test material by using TonEBP antibody.

The measurement method may also include measuring amounts of TonEBP and LCN2 proteins by further using LCN2 antibody in addition to the TonEBP antibody, but it is not limited thereto.

As for the test sample to be used for the aforementioned measurement method, any one selected from the group consisting of blood serum, blood plasma, and blood may be used. Preferably, blood serum is used as a test sample, but it is not limited thereto.

Moreover, the measurement method may be carried out by characteristically including steps of: measuring the level of TonEBP protein in a test sample of human body fluid; and determining the enhanced TonEBP protein compared to normal control group. More preferably, the test sample of body fluid may be blood plasma which is collected from a testee for diagnosis of mild cognitive impairment.

Hereinbelow, the present invention is explained in greater detail in view of the Examples. However, the following Examples are given only for specific explanation of the present invention and it would be evident to a person who has common knowledge in the pertinent art that the scope of the present invention is not limited by them.

### EXAMPLES

### Example 1. Establishment of Subject Group with Diabetes and Cognitive Impairment and Determination of TonEBP and LCN2 in Blood

### 1) Subject Selection for Research and Data Collection

For the test, elderlies in the dementia cohort established by National Dementia Research Group of Chosun University were chosen and data were collected through in-depth examination. All the examination and data were obtained from research subjects who had agreed to the research after being asked for the agreement.

Based on selective examination, data for demographic information (i.e., gender, age, education, employment, and marital status), basic physical examination (i.e., height, body weight, body mass index, and blood pressure), and family health history (i.e., dementia, cerebral disease, and diabetes) were obtained. In addition, at the pre-examination stage, research subjects were classified through K-MMSE (Korean version of Mini-Mental State Examination) as a simple screening test for dementia.

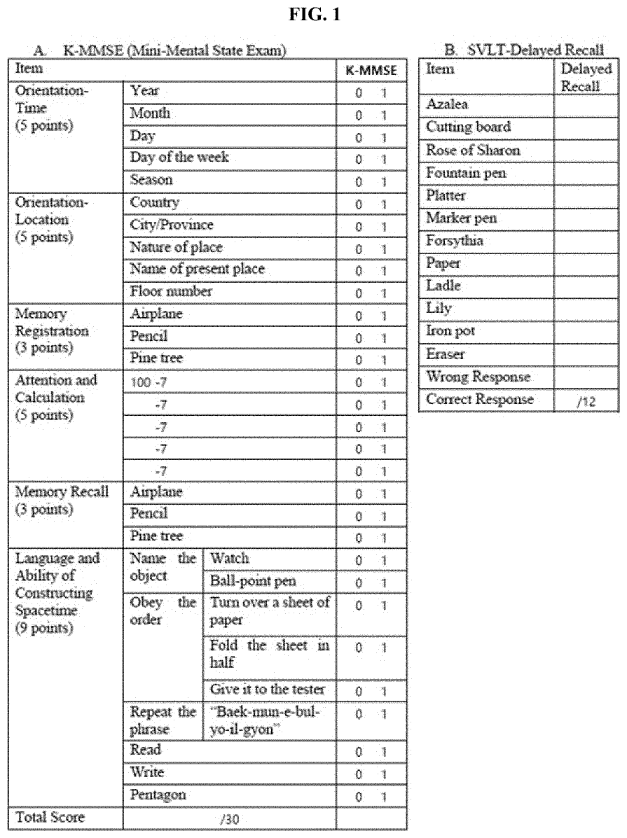

As for the K-MMSE test, a simple cognitive function test was carried out in terms of Orientation-Time (full-score; 5 points), Orientation-Location (full-score; 5 points), Memory Registration (full-score; 3 points), Attention and Calculation (full-score; 5 points), Memory Recall (full-score; 3 points), and Language and Ability of Constructing Spacetime (full-score; 9 points), i.e., 30 points in total. The test sheet is the same as illustrated in A of FIG. 1.

Moreover, based on SNSB (Seoul Neuropsychological Screening Battery) including SVLT-delayed recall (SVLT-delayed recall) at the in-depth examination stage, data of in-depth neurological and psychological test were obtained.

As for the SVLT-delayed recall, 12 words shown in B of FIG. 1 were read, number of the recalled word was expressed as a score (carried out 3 times in total), and, 20 minutes thereafter, number of the still-recalled word was added to the score of the measurement (total score; 12 points).

Moreover, based on a blood test, examination data of HbA1C, cholesterol, triglyceride, AST (aspartate aminotransferase), ALT (alanine aminotransaminase), LDL-cholesterol, HDL-cholesterol, and the like were obtained.

### 2) Classification of Groups

Based on the BMI and HbA1C measured as described in the above, classification was made between the normal and diabetic patients. People having 3 points or less and 26 points or less as the SVLT-delayed recall score and K-MMSE score, respectively, were classified as those having mild cognitive impairment (MCI).

Standard values of BMI and HbA1C are described in the following Table 1 and Table 2.

TABLE 1
Standard of NIH of USA, WHO and Asian Obesity Association

| | | Standard of Asian |
|---|---|---|
| | NIH, WHO Standard | Obesity Association |
| Classification | BMI (kg/m²) | BMI (kg/m²) |
| Under weight | 18.5 or less | 18.5 or less |
| Normal range | more than 18.5-24.9 or | more than 18.5-22.9 or |
| | less | less |
| Pre-obesity | 25 or more-29.9 or less | 23 or more-24.9 or less |
| 1st Stage obesity | 30 or more-34.9 or less | 25 or more-29.9 or less |
| 2nd Stage obesity | 35 or more-39.9 or less | 30 or more |
| 3rd Stage obesity | 40 or more | |

TABLE 2
HbA1C Standard

| | HbA1c test |
|---|---|
| Normal | Less than 5.65% |
| Pre-diabetes | 5.65% or more-less than 6.5% |
| Diabetes | 6.5% or more |

### 3) Data Values Measured for Each Group

Data values measured for each of three different groups in total, i.e., normal (CTL); obesity and diabetes (DM); obesity, diabetes and mild cognitive impairment (DM-MCI), which have been classified according to the above classification of groups, are described in the following Table 3.

TABLE 3

| | CTL | DM | DM-MCI |
|---|---|---|---|
| | (n = 28) | (n = 27) | (n = 28) |
| Age (years) | 72.3 ± 0.74 | 72.63 ± 0.85 | 74.87 ± 0.8 |
| BMI (kg/m²) | 22.29 ± 0.29 | 25.64 ± 0.49 | 25.32 ± 0.39 |
| HbA1C (%) | 5.32 ± 0.03 | 7.24 ± 0.17 | 7.28 ± 0.19 |
| education | 13.5 ± 0.8 | 13.97 ± 0.8 | 13.87 ± 0.74 |
| cholesterol (mg/dL) | 188.17 ± 6.05 | 166.9 ± 7.04 | 172.1 ± 7.44 |
| triglyceride (mg/dL) | 95.9 ± 11.28 | 112.17 ± 9.99 | 135.27 ± 12.96 |
| AST (IU/L) | 21.7 ± 0.94 | 22.96 ± 1.68 | 24.5 ± 1.53 |
| ALT (IU/L) | 15.66 ± 1.23 | 20.82 ± 1.72 | 25.15 ± 2.28 |
| BP systolic (mmHg) | 121.7 ± 2.42 | 127.73 ± 2.94 | 131.07 ± 4.4 |
| BP diastolic (mmHg) | 71.1 ± 1.59 | 70.47 ± 1.94 | 73.5 ± 2.57 |
| LDLcholesterol | 119.2 ± 4.87 | 99.17 ± 6.42 | 106.85 ± 6.92 |
| (mg/dL) | | | |
| HDLcholesterol | 55.08 ± 2.5 | 49.42 ± 2.66 | 47.43 ± 2.45 |
| (mg/dL) | | | |
| SVLT-Delayed recall | 6.07 ± 0.36 | 5.3 ± 0.38 | 2.9 ± 0.29 |
| K-MMSE score | 28.43 ± 0.23 | 28.13 ± 0.24 | 25.93 ± 0.4 |

### 4) Determination of TonEBP and LCN2 in Blood

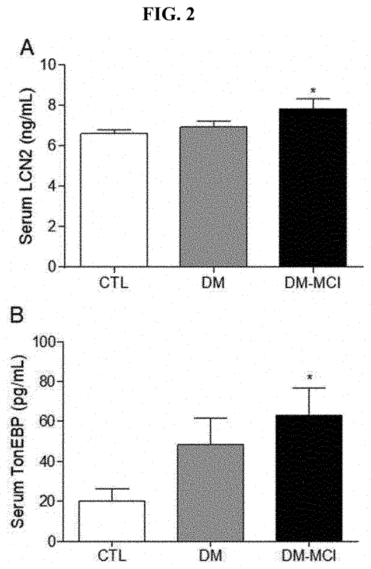

Protein concentration of TonEBP and LCN2 in blood was determined for the groups of normal (CTL); obesity and diabetes (DM); obesity, diabetes and mild cognitive impairment (DM-MCI). As the result is shown in FIG. 2, it was found that TonEBP and LCN2 protein concentration is significantly higher in blood from DM-MCI group compared to the normal group, and it is higher even compared to DM group.

### Example 2. Determination of Correlation Between Cognitive Impairment and TonEBP Protein in Animal Model Induced to have Obesity and Diabetes

To determine any correlation between cognitive impairment and TonEBP protein in an animal model which has been induced to have obesity and diabetes, a three-week old male C57BL/6 mouse was obtained from Central Experimental Animals. To prepare an obese animal model, the mouse was put on high fat diet (HFD, 60 kcal % fat, Research Diet, USA) for 20 weeks. To prepare a mouse with typical Type 2 diabetes, 16 weeks after the high fat diet, the mouse was intraperitoneally injected once with streptozotocin (STZ, 100 mg/kg) followed again by high fat diet for 4 weeks. Body weight was measured, after the first measurement on the start day of test, for 20 weeks with an interval of 4 weeks. The animal was then sacrificed, and liver tissues, epididymal fat, perirenal fat, and mesentery fat were collected from the animal and weighed, respectively.

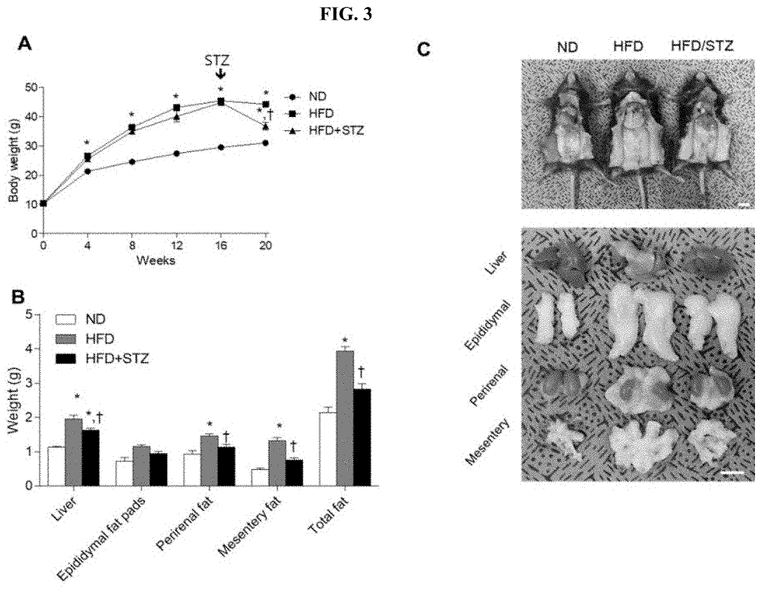

As the result is shown in FIG. 3, it was found that the body weight increased more in the group induced to have obesity by high fat diet (HFD) compared to the normal diet group (ND), and, in the group induced to have obesity and diabetes by high fat diet and a treatment of streptozotocin (HFD+STZ), it was shown that the body weight tends to increase like HFD but it decreases upon the administration of streptozotocin. In particular, weight of the abdominal fat tissues including liver was lower compared to HFD group.

After twenty weeks, the above ND group, HFD group and HFD+STZ group were subjected to fasting for 12 hours. Then, blood was taken from tail vein to measure blood sugar. After the measurement of blood sugar, the animal was anesthetized. Blood was taken from the animal heart and centrifuged for 15 minutes at 4° C., 3000 rpm to separate blood serum. Thus-separated blood serum was kept at −80° C. until the use for experiment.

Concentration of LCN2 and TonEBP proteins in the blood serum was measured by using mouse lipocalin-2/NGAL Quantikine ELISA kit (No; MLCN20, R&D systems) and mouse NFATS ELISA kit (No; E3089m, Wuhan EIAab Science) according to the kit protocols.

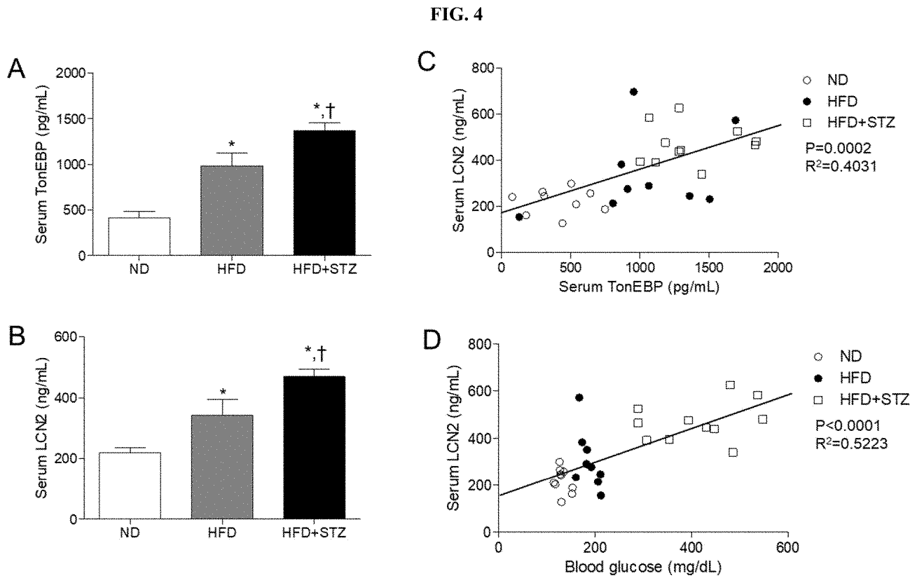

As the result is shown in FIG. 4, the blood concentration of TonEBP and LCN2 proteins became higher as a result of high fat diet. According to administration of streptozotocin, the blood concentration of TonEBP and LCN2 proteins became even higher.

### Example 3. Morris Water Maze Test for Examining Long Term Memory

To examine long term memory, Morris water maze test was carried out.

In water maze pool with diameter of 100 cm equipped with a round escape platform, water (21±2° C.) was filled 1 cm high above the round escape platform such that the platform is not visible. By dissolving instant coffee cream in the pool, it was made sure that the round escape platform is in invisible state. The pool was divided into 4 quadrants with same size, i.e., northeast (NE), northwest (NW), southeast (SE), and southwest (SW). Among them, at the center of southeast (SE) quadrant, the round escape platform was placed, and, from the quadrant rim, the mouse was allowed to enter the water while facing the wall. For 4 days, the mouse was allowed to enter the pool for 90 seconds, 4 times a day, and the time required for the mouse to climb onto the round escape platform (i.e., escape latency) was measured. For the mouse not able to climb onto the round escape platform even after 90 seconds, location of the round escape platform was shown to the mouse, which was then allowed to stay thereon for 20 seconds. For probe test, on Day 5 as the last day, the mouse was forced to swim for 60 seconds by removing the round escape platform, and the time for the mouse to spend in the area of round escape platform (i.e., time in the target platform quadrant) and the number of the round escape platform crossings by the mouse (i.e., number of the platform crossings) were measured. Every behavior of the mouse in water maze was recorded by using a video tracking system. (Noldus EthoVision XT7, Noldus Information Technology, The Netherlands).

Upon the termination of the test, to extract proteins from brain hippocampal tissues of the animal model, T-PER tissue protein extraction reagent added with proteinase inhibitor was added to the tissues. After homogenization and centrifugation of the homogenates, protein quantification was carried out. 15 μg of the protein was separated by 10% (w/v) SDS-PAGE, subjected to electroblotting onto a PVDF membrane, and, after blocking with 5% (w/v) skim milk, treated with anti-LCN2 and anti-TonEBP, which are primary antibodies. After that, following the treatment with secondary antibodies conjugated with horseradish peroxidase, the proteins were detected by chemiluminescence.

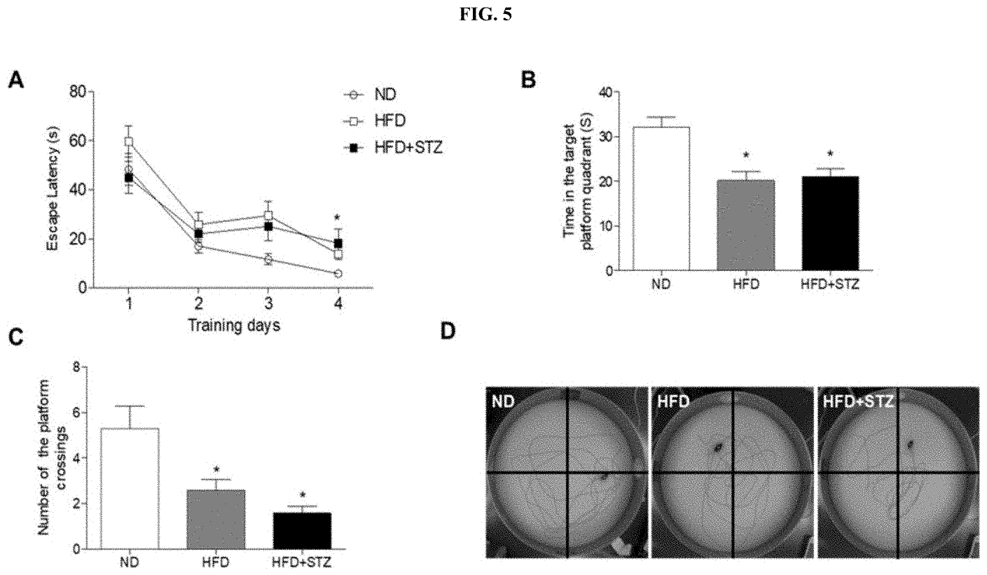

As the result is shown in FIG. 5, the time required for the mouse to climb onto the round escape platform after entering the water (i.e., escape latency) was longer in the group induced to have obesity by high fat diet (HFD) and also in the group induced to have obesity and diabetes by high fat diet and a treatment of streptozotocin (HFD+STZ) compared to the normal diet group (ND). In addition, the time for the mouse to spend in the area of round escape platform (i.e., time in the target platform quadrant) after forcing the mouse to swim for 60 seconds following the removal of round escape platform and the number of the round escape platform crossings by the mouse (i.e., number of the platform crossings) were lower in HFD group and HFD+STZ group compared ND, indicating that the HFD group and HFD+STZ group have impaired cognitive function. It was also found that the cognitive function is impaired more in HFD+STZ group, in particular.

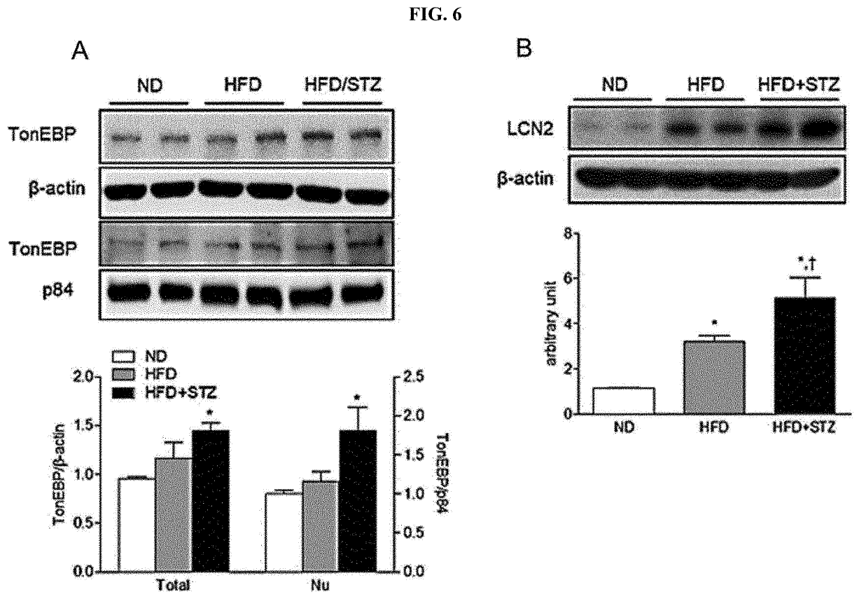

As illustrated in FIG. 6, it was also found that, compared to the normal diet group (ND), the expression of TonEBP and LCN2 is higher in hippocampal tissues of HFD group and HFD+STZ group with impaired cognitive function. To determine the nuclear expression of TonEBP which is known to be functional in nucleus, nucleus was isolated from the cells of hippocampal tissues, nuclear proteins were isolated, and the expression of TonEBP was examined. As a result, it was found that, compared to the normal diet group (ND), HFD group and HFD+STZ group with impaired cognitive function have significantly increased expression of TonEBP. It was also found that the expression of TonEBP and LCN2 is significantly higher in HFD+STZ group, in particular, compared to HFD group.

Based on the above results, it was recognized that the expression of TonEBP and LCN2 proteins is enhanced in hippocampal tissues of an obese or diabetic mouse with impaired cognitive function.

### Example 4. Expression of TonEBP and LCN2 Proteins in Ob/Ob Mouse as Obesity and Type 2 Diabetes Animal Model

To determine the expression of TonEBP and LCN2 proteins in ob/ob mouse which is frequently used as an animal model with obesity and Type 2 diabetes caused by mutation of leptin gene, a five-week old male ob/ob mouse was obtained from Central Experimental Animals and kept for 20 weeks while maintaining suitable temperature (22±2° C.) and 12-hour light and dark cycle and allowing free access to food and water. As a control, a five-week old male C57BL/6 mouse having no mutation of leptin gene was used.

Twenty weeks thereafter, the animal was anesthetized and blood was taken from the heart and centrifuged for 15 minutes at 4° C., 3000 rpm to separate blood serum. Thus-separated blood serum was kept at −80° C. until the use for experiment. LCN2 concentration in blood serum was measured by using mouse lipocalin-2/NGAL Quantikine ELISA kit (No; MLCN20, R&D systems).

The animal was fasted at least for 12 hours before collecting the blood. From the tail vein, the blood was taken, and blood glucose was measured for each group by using blood glucose tester (Accu-Check). Blood serum LCN2 and a change in blood glucose, and correlation between them were analyzed by using Graphpad Prism 5.

In addition, to extract proteins from brain hippocampal tissues of the animal model, T-PER tissue protein extraction reagent added with proteinase inhibitor was added to the tissues. After homogenization and centrifugation of the homogenates, protein quantification was carried out. 15 μg of the protein was separated by 10% (w/v) SDS-PAGE, subjected to electroblotting onto a PVDF membrane, and, after blocking with 5% (w/v) skim milk, treated with anti-LCN2 and anti-TonEBP, which are primary antibodies. After that, following the treatment with secondary antibodies conjugated with horseradish peroxidase, the proteins were detected by chemiluminescence.

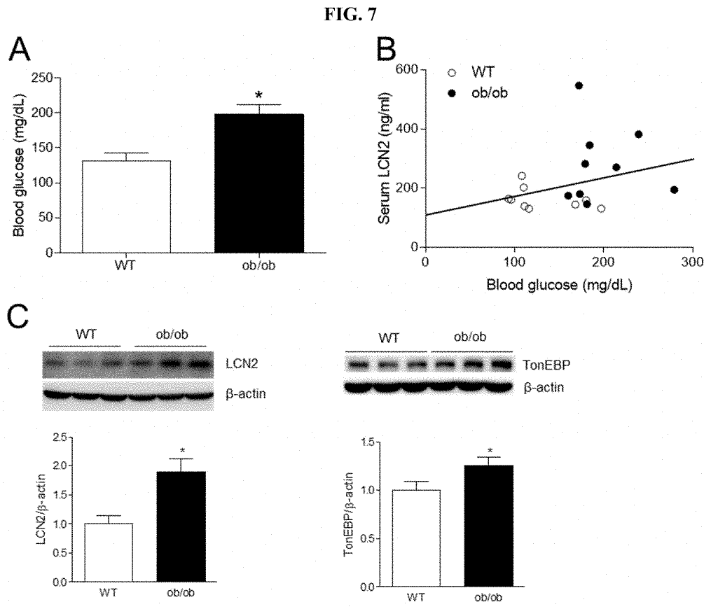

As the result is shown in FIG. 7, it was found that increased blood glucose is obtained from the ob/ob mouse, and there is a directly proportional relationship between the blood glucose and LCN2 protein in blood. It was also found that higher amounts of LCN2 and TonEBP proteins are present in the hippocampal tissues of ob/ob mouse compared to the normal mouse.

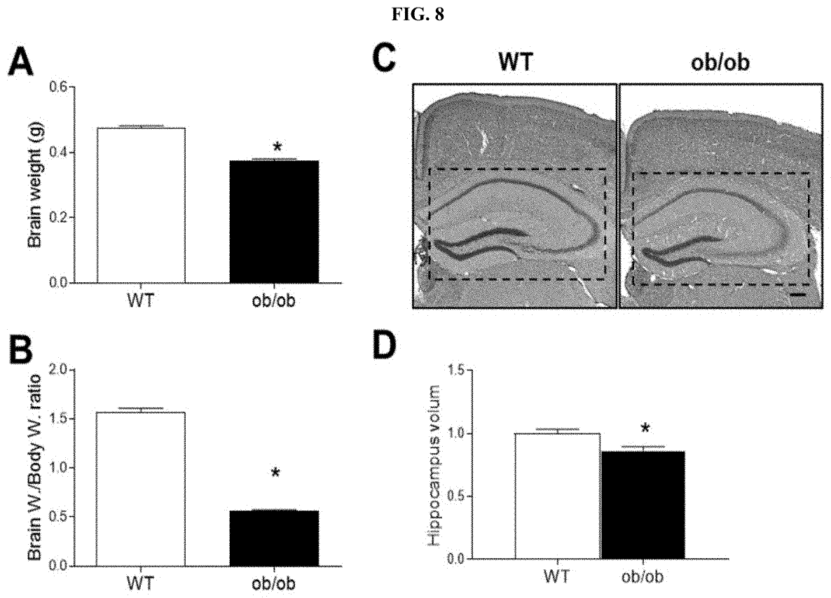

In addition, as a result of measuring the weight of mouse brain after the removal as shown in A and B of FIG. 8, it was found that the brain weight of ob/ob mouse is less than the normal mouse, and such decrease is more significant when the result is converted in terms of the ratio relative to body weight.

In addition, as illustrated in C and D of FIG. 8, mouse brain tissues were sliced to thickness of 30 μm, stained with hematoxylin-eosin, and subjected to observation under an optical microscope. As a result of calculating and comparing the area of hippocampus region, it was found that the volume of hippocampus, which is related to memory and learning, is reduced in the ob/ob mouse compared to the normal group.

Based on the above result, it was recognized that the functional problem of a hippocampus region is related with the enhanced expression of LCN2 and TonEBP proteins in hippocampal tissues.

1-8. (canceled)

9. A method for diagnosing mild cognitive impairment, the method comprising:
obtaining a sample from a subject to be diagnosed; and
measuring a level of tonicity-responsible enhancer binding protein (TonEBP) in the sample by using TonEBP antibody to determine if the level of the TonEBP is above a first predetermined level.

10. The method of claim 9, wherein the measuring further comprises measuring a level of lipocalin-2 (LCN2) in the sample by using LCN2 antibody to determine if the level of the LCN2 is above a second predetermined level.

11. The method of claim 9, wherein the TonEBP antibody is a monoclonal antibody.

12. The method of claim 9, wherein the TonEBP antibody is a polyclonal antibody.

13. The method of claim 9, wherein the mild cognitive impairment is caused by obesity or diabetes.

14. The method of claim 9, wherein the sample is obtained from at least one selected from the group consisting of blood serum, blood plasma, and blood.
